(19) United States
(12) Patent Application Publication
(10) Pub. No.: US 2016/0324562 A1
(43) Pub. Date: Nov. 10, 2016

(54) ORTHOPEDIC DRIVER INSTRUMENT AND METHODS OF PRODUCTION

(71) Applicant: SMITH & NEPHEW, INC., Memphis, TN (US)

(72) Inventors: Charles R. Baker, Lakeland, TN (US); Gene E. Austin, Bartlett, TN (US)

(73) Assignee: SMITH & NEPHEW, INC., Memphis, TN (US)

(21) Appl. No.: 15/107,794

(22) PCT Filed: Dec. 23, 2014
(86) PCT No.: PCT/US14/72155

§ 371 Date: Jun. 23, 2016

Related U.S. Application Data
(60) Provisional application No. 61/920,315, filed on Dec. 23, 2013.

(51) Int. Cl. A61B 17/88; B29C 45/14; B22F 3/22

(52) U.S. Cl. CPC A61B 17/8888; B22F 3/225; B29C 45/14336; B29K 2023/06; B29L 2031/283

(57) ABSTRACT

An orthopedic driver instrument including a metal injection molded driver bit having an overall bit length, and a plastic injection molded handle having an overall handle length at least twice the overall bit length, the handle including a gripping portion and an elongate shaft portion over molded about a shank portion of the driver bit. A method of producing an orthopedic driver instrument is also provided including forming a metallic driver bit using a metal injection molding process, and forming a plastic handle using a plastic injection molding process, wherein the plastic handle has a gripping portion and an elongate shaft portion, and the plastic injection molding process comprises over molding the elongate shaft portion of the plastic handle about a shank portion of the metallic driver bit. In some embodiments, the overall length of the plastic handle is at least twice the overall length of the metallic driver bit.

### CROSS REFERENCE TO RELATED APPLICATIONS

This application claims the benefit of U.S. Provisional Application No. 61/920,315 filed Dec. 23, 2013, the contents of which are incorporated herein by reference in their entirety.

### FIELD OF THE INVENTION

The present invention relates generally to orthopedic instruments for use in orthopedic surgeries or procedures, and more particularly but not exclusively relates to an orthopedic driver instrument and methods of producing the same.

### BACKGROUND

Orthopedic drivers are commonly used to drive bone screws or other types of fasteners into bone and/or into engagement with other structures or devices such as, for example, orthopedic bone plates or other types of implants. In some instances, the orthopedic driver is used in a single surgery or procedure and is then discarded, thereby eliminating the need to clean and sterilize the driver for subsequent use in another surgery or procedure. However, the manufacturing/fabrication costs associated with producing a driver must be taken into consideration when disposing of the driver after a single use. In the past, orthopedic drivers have been manufactured/fabricated under relatively tight tolerance levels (i.e., via precise machining processes and techniques), and have been made of exotic, ultra-durable, autoclavable materials and subjected to specialized heat treatment procedures. As should be appreciated, these factors all tend to increase manufacturing/fabrication costs, thereby resulting in a relatively expensive orthopedic driver. Significant reductions in the costs associated with producing an orthopedic driver are required in order to economically justify disposal of the driver after a single use.

Thus, there remains a need to provide an improved orthopedic driver instrument and methods of producing the same. The present invention satisfies this need and provides other benefits and advantages in a novel and unobvious manner.

### SUMMARY

While the actual nature of the invention covered herein can only be determined with reference to the claims appended hereto, certain forms of the invention that are characteristic of the embodiments disclosed herein are described briefly as follows.

In general, a novel orthopedic driver instrument is provided along with methods of producing the orthopedic driver instrument.

In one aspect of the invention, a method of producing an orthopedic driver instrument is provided including: forming a metallic driver bit using a metal injection molding process; and forming a plastic handle using a plastic injection molding process, wherein the plastic handle has a gripping portion and an elongate shaft portion extending axially from the gripping portion, and wherein the plastic injection molding process comprises over molding the elongate shaft portion of the plastic handle about a shank portion of the metallic driver bit.

In another aspect of the invention, a method of producing an orthopedic driver instrument is provided including: forming a metallic driver bit using a metal injection molding process, the metallic driver bit having an overall bit length; and forming a plastic handle over a portion of the metallic driver bit using a plastic injection molding process, and wherein the plastic handle has an overall handle length that is at least twice the overall bit length.

In a further aspect of the invention, an orthopedic driver instrument is provided including a metal injection molded driver bit having an overall bit length, and a plastic injection molded handle having an overall handle length that is at least twice the overall bit length, wherein the handle includes a gripping portion and an elongate shaft portion extending axially from the gripping portion, and wherein the elongate shaft portion is over molded about a shank portion of the metallic driver bit.

It is one object of the present invention to provide an improved orthopedic driver instrument and methods of producing the same. Further embodiments, forms, features, aspects, benefits, objects, and advantages of the present invention will become apparent from the detailed description and figures provided herewith.

### BRIEF DESCRIPTION OF THE DRAWINGS

FIG. 1 is a front/top, right-side perspective view of an orthopedic driver instrument according to one embodiment of the present invention.

FIG. 2 is a front/top, left-side perspective view of the orthopedic driver instrument of FIG. 1.

FIG. 3 is a front view of the orthopedic driver instrument of FIG. 1.

FIG. 4 is a top view of the orthopedic driver instrument of FIG. 1.

FIG. 5 is a right-side view of the orthopedic driver instrument of FIG. 1.

FIG. 6 is a left-side view of the orthopedic driver instrument of FIG. 1.

FIG. 7 is a front/top, right-side perspective view of another embodiment of the orthopedic driver instrument of FIG. 1.

### DESCRIPTION OF THE ILLUSTRATED EMBODIMENTS

For the purpose of promoting an understanding of the principles of the present invention, reference will now be made to the embodiments illustrated in the drawings and specific language will be used to describe the same. It will nevertheless be understood that no limitation of the scope of the invention is hereby intended. Any alterations and further modifications in the described embodiments, and any further applications of the principles of the invention as described herein are contemplated as would normally occur to one skilled in the art to which the invention relates. The following descriptions and illustrations of non-limiting forms and embodiments of the present invention are exemplary in nature, it being understood that the descriptions and illustrations related thereto are in no way intended to limit the inventions disclosed herein and/or their applications and uses.

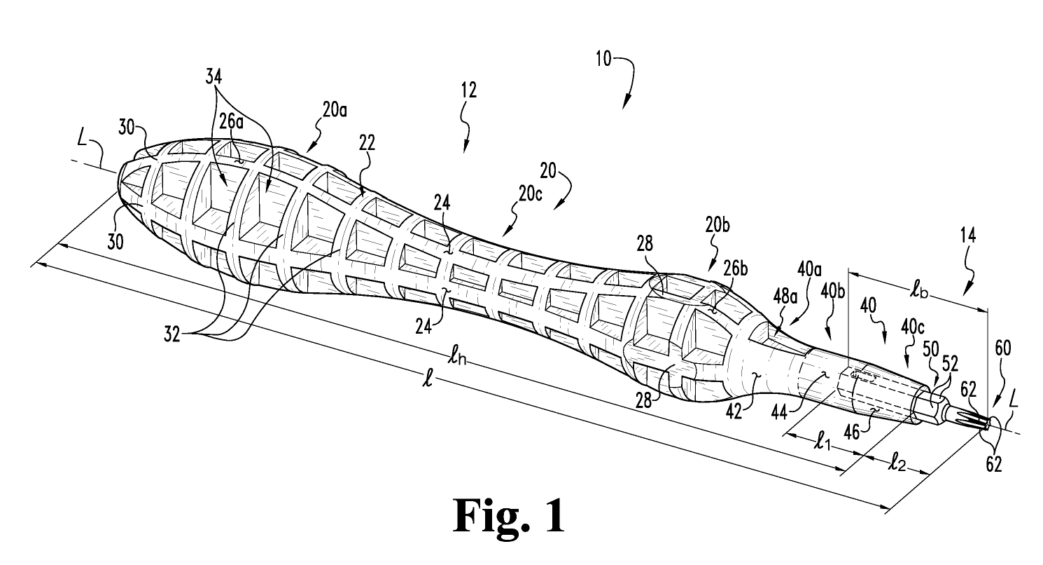

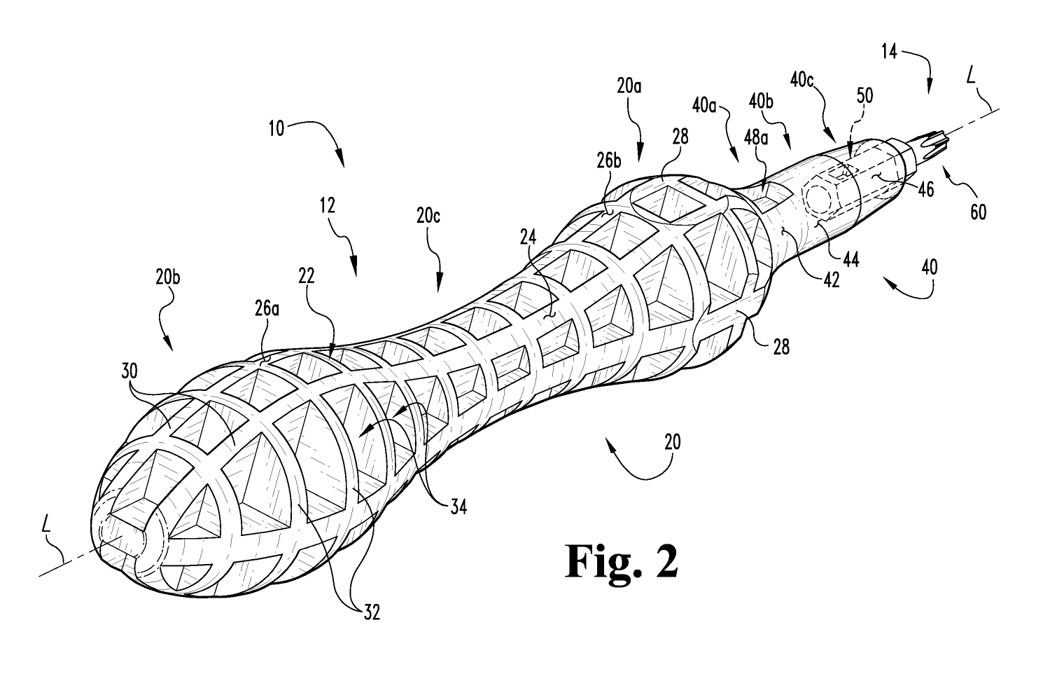

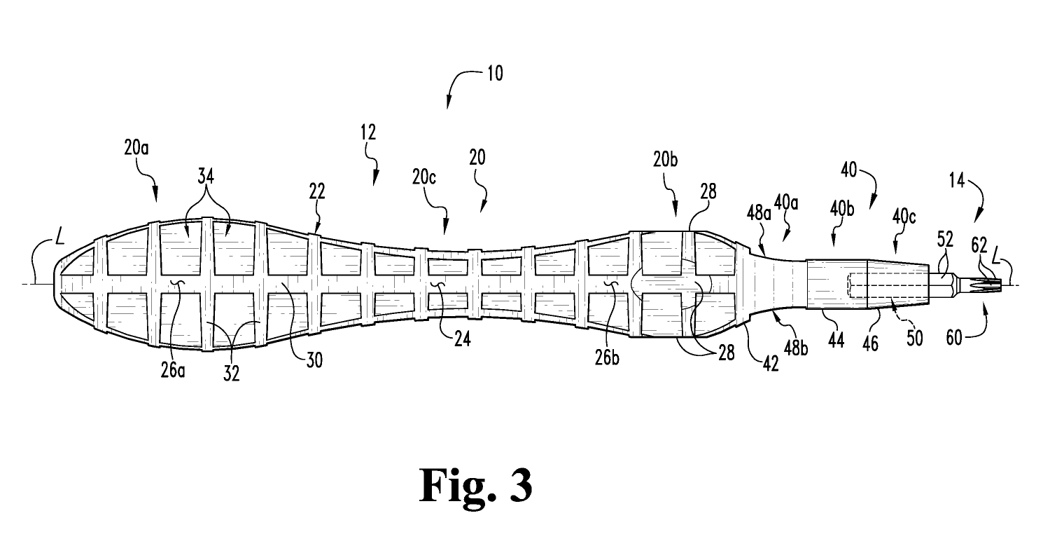

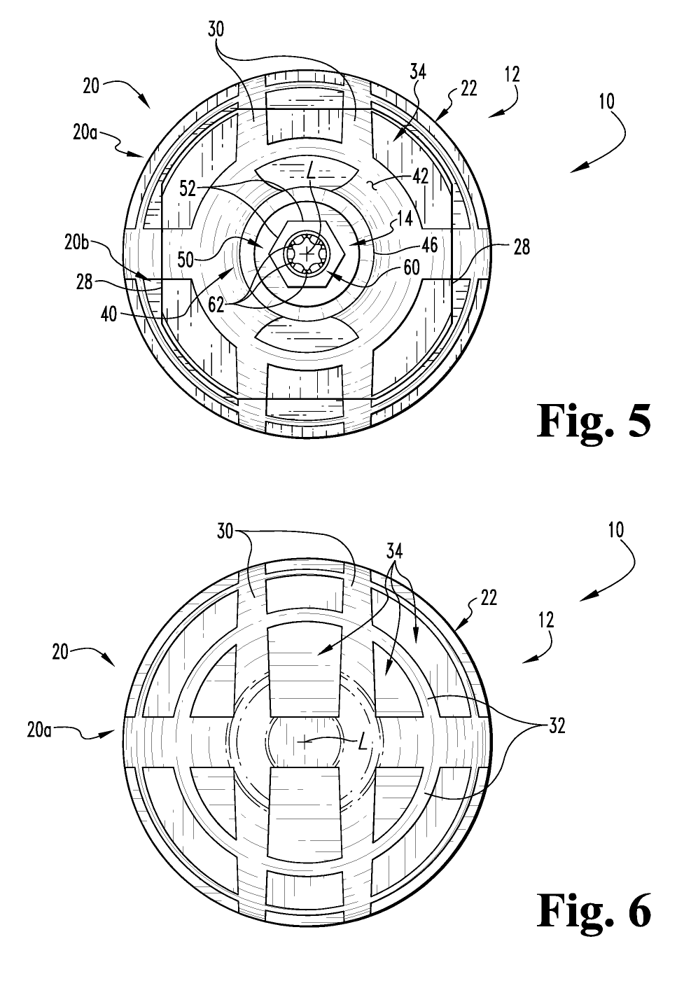

Referring to FIGS. 1-6, shown therein is an orthopedic driver instrument 10 according to one form of the invention. The driver instrument 10 has an overall length extending along a longitudinal axis L, and generally includes a proximal handle 12 and a distal driver tip or bit 14. In one embodiment, the driver instrument 10 is configured for use in association with orthopedic surgical procedures to manipulate and drive bone screws or other types of bone anchors into bone. As will be discussed below, the driver instrument 10 includes a distal end portion that is releasably engagable with the head of a bone screw to manipulate the bone screw to an anchor location or surgical site for driving engagement into bone. However, it should be appreciated that the orthopedic driver instrument 10 may be used in association with a variety of orthopedic surgeries or procedures, and may be used to manipulate various types and configurations of bone anchors and/or other orthopedic devices including bone shaping/cutting devices.

The proximal handle 12 and the distal bit 14 of the driver instrument 10 are formed of biocompatible materials including, for example, plastic materials such as polyethylene or other polymeric materials, and metallic materials such as stainless steel or titanium. However, other suitable biocompatible materials are also contemplated including other types of plastic or polymeric materials, other types of metallic materials, and/or composite materials. In one specific embodiment, the proximal handle 12 is made of a plastic material, and the distal bit 14 is made of a metallic material. However, it should be appreciated that other combinations of materials are also contemplated. In one aspect of the invention, the proximal handle 12 is made of a plastic injection molded material, and is formed via a plastic injection molding process. In another aspect of the invention, the distal bit 14 is made of a metal injection molded material, and is formed via a metal injection molding process. However, it should be understood that other types of materials and formation processes or manufacturing techniques are also contemplated for use in association with the present invention.

In the illustrated embodiment, the proximal handle 12 includes a gripping portion or main body 20, and a stem portion or elongate shaft 40 extending axially from the gripping portion 20. Additionally, the distal bit 14 includes a shank or attachment portion 50, and a shaped end or engagement portion 60 configured for engagement with a bone anchor such as, for example, the head of a bone screw or other types of fastener devices. In one embodiment, the stem portion 40 of the proximal handle 12 is over molded about the shank portion 50 of the distal bit 14. In this manner, the proximal handle 12 and the distal bit 14 are integral with one another so as to define a unitary driver instrument 10 wherein the distal bit 14 is permanently attached/engaged to the proximal handle 12. However, other embodiments are also contemplated wherein the proximal handle 12 and the distal bit 14 are detachable/disengageable from one another.

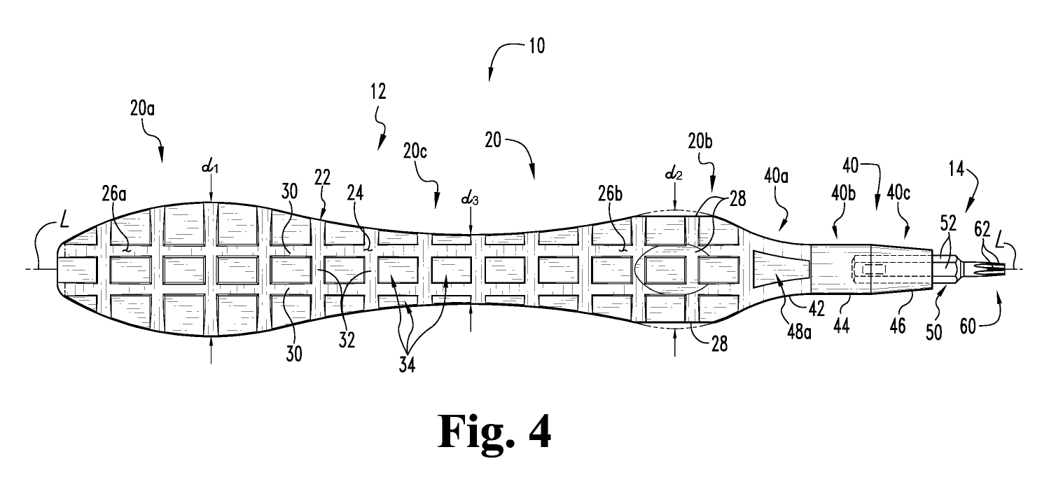

In the illustrated embodiment of the proximal handle 12, the gripping portion 20 extends generally along the longitudinal axis L and includes a proximal region 20a, a distal region 20b, and a central region 20c extending between the proximal and distal regions 20a, 20b. The gripping portion 20 is sized, shaped and configured to provide an ergonomic design that is readily grasped and manipulated by a user (i.e., a surgeon). In one embodiment, the gripping portion 20 has an outer surface 22 defining a longitudinally-extending concave surface contour 24 extending along the central region 20c, and longitudinally-extending convex surface contours 26a, 26b extending along the proximal and distal regions 20a, 20b, respectively, and arranged on opposite sides of the longitudinally-extending concave surface contour 24. In another embodiment, the transitions between the proximal, distal and central regions 20a, 20b and 20c are smooth so as to avoid sharp corners or abrupt transitions between the regions of the handle gripping portion 20. As illustrated in FIG. 4, the proximal region 20a defines a maximum outer diameter d₁, the distal region 20b defines a maximum outer diameter d₂, and the central region 20c defines a minimum outer diameter d₃. In one embodiment, the minimum outer diameter d₃ of the central region 20c is less than both the maximum outer diameters d₁ and d₂ of the proximal and distal regions 20a, 20b. In another embodiment, the maximum outer diameter d₁ of the proximal region 20a is greater than the maximum outer diameter d₂ of the distal region 20b. However, other configurations of the gripping portion 20 are also contemplated, including embodiments where the maximum outer diameters d₁ and d₂ of the proximal and distal regions 20a, 20b are substantially equal to one another.

In some embodiments of the driver instrument 10, the outer surface 22 of the gripping portion 20 further defines a plurality of flats or flattened regions 28 dispersed along and/or about the distal longitudinally-extending convex surface contour 26b of the distal region 20b. In the illustrated embodiment, the outer surface 22 defines four flattened regions 28 dispersed uniformly about a circumference of the distal region 20b of the gripping portion 20. However, other embodiments are also contemplated wherein flattened regions may be located along other regions of the gripping portion 20 including, for example, the proximal longitudinally-extending convex surface contour 26a of the proximal region 20a. It should be appreciated that the size of the flattened regions 28 may vary, and that any number of the flattened regions 28 may be dispersed along and/or about various regions of the gripping portion 20, including embodiments of the driver instrument 10 that do not include any of the flattened regions 28.

Additionally, in the illustrated embodiment, the gripping portion 20 includes a plurality of longitudinally-extending ribs 30 and a plurality of transversely-extending ribs 32 extending between adjacent pairs of the longitudinally-extending ribs 30 so as to define a grid pattern. The longitudinally-extending ribs 30 cooperate with the transversely-extending ribs 32 to define a plurality of hollow recessed regions or indentations/depressions 34 dispersed along the length of the gripping portion 20 and about the circumferential periphery of the gripping portion 20, thereby providing the gripping portion 20 with a hollow grid pattern along its length and about its perimeter. As should be appreciated, the longitudinally-extending ribs 30, the transversely-extending ribs 32, and the recesses 34 cooperate to provide the gripping portion 20 with a frictional non-slip configuration to facilitate secure grasping and handling of the proximal handle 12 by the surgeon or other medical personnel. As should be further appreciated, the ribbed configuration of the proximal handle 12 defining the hollow grid pattern significantly reduces the amount of material required to form the proximal handle 12, and does so without a significant reduction in the strength and structural integrity of the proximal handle 12. The ribbed configuration and hollow grid pattern of the gripping portion 20 also reduce the overall weight of the driver instrument 10.

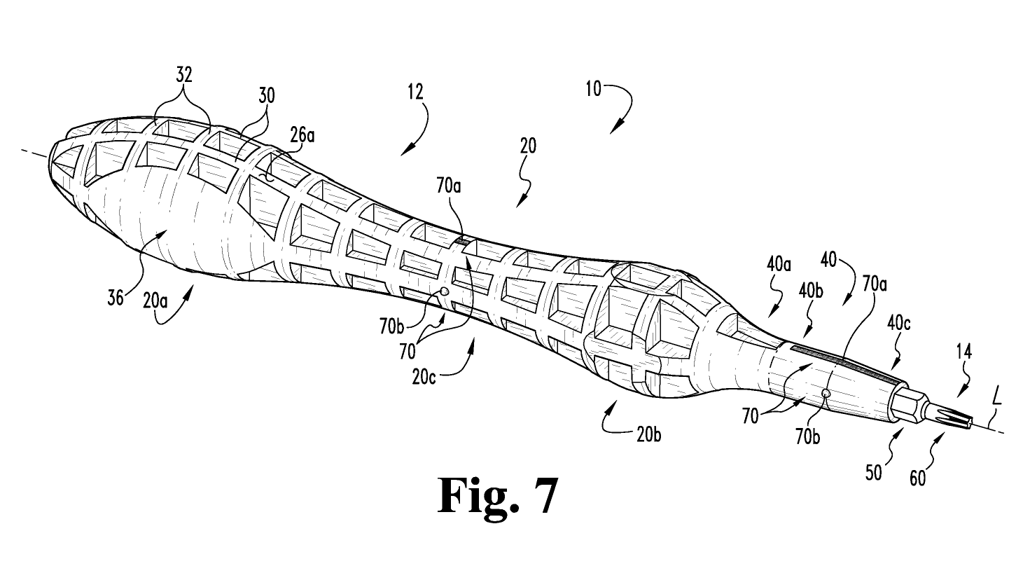

In another embodiment of the driver instrument 10 illustrated in FIG. 7, the gripping portion 20 of the proximal handle 12 may be provided with one or more non-ribbed regions 36 located along and about one or more regions of the gripping portion 20 including, for example, the proximal longitudinally-extending convex surface contour 26a. In the illustrated embodiment, the non-ribbed regions 36 define the same localized curvature as the adjacent ribbed regions of the gripping portion 20 which include the longitudinally-extending ribs 30 and the transversely-extending ribs 32, thereby providing the proximal handle 12 with a substantially continuous and uniform outer surface. As should be appreciated, the non-ribbed regions 36 provide the gripping portion 20 with a relatively smooth, uninterrupted gripping surface to promote comfortable handling and rotation of the driver instrument 10 by the surgeon, particularly when driving a large number of bone screws or fasteners into bone. Additionally, in the illustrated embodiment, the gripping portion 20 defines a pair of the non-ribbed regions 36 located on opposite sides of the proximal handle 12. However, it should be appreciated that the gripping portion 20 may be provided with any number of the non-ribbed regions 36, and that the non-ribbed regions 36 may be dispersed along and/or about various regions of the gripping portion 20.

In the illustrated embodiment of the proximal handle 12, the stem portion 40 extends from the gripping portion 20 generally along the longitudinal axis L, and the stem portion 40 and the gripping portion 20 together form a monolithic, single-piece handle structure. The stem portion 40 generally includes a proximal transition region 40a extending axially from the distal end of the gripping portion 20, a central region 40b extending axially from the proximal transition region 40a, and a distal region or end portion 40c extending axially from the central region 40b. However, it should be appreciated that other shapes and configurations of the stem portion 40 are also contemplated.

In one embodiment, the proximal transition region 40a is conically-shaped and has an outer concave surface 42 extending along the longitudinal axis L and defining an inward taper in a proximal-distal direction. In another embodiment, the central region 40b is cylindrically-shaped and has an outer cylindrical surface 44 defining a maximum outer diameter d₄. In a further embodiment, the distal region or end portion 40c is conically-shaped and has an outer conical surface 46 extending along the longitudinal axis L and also defining an inward taper in a proximal-distal direction. However, other shapes, sizes and configurations of the proximal transition region 40a, the central region 40b, and the distal region 40c are also contemplated.

In one specific embodiment, the maximum outer diameter d₄ of the central region 40b is less than both the maximum outer diameters d₁ and d₂ of the proximal and distal regions 20a, 20b of the gripping portion 20. In another specific embodiment, the maximum outer diameter d₄ of the central region 40c is less than or substantially equal to the minimum outer diameter d₃ of the central region 20c of the gripping portion 20. However, it should be appreciated that other embodiments are also contemplated where the relative size of the central region 40b of the stem portion 40 varies relative to the regions of the gripping portion 20.

Additionally, the stem portion 40 may include a pair of recesses or indentations 48a, 48b extending along the length of the proximal transition portion 42 and positioned on opposite sides of the stem portion 40. However, other embodiments are also contemplated where any number of the recesses or indentations may be provided along/about any region of the stem portion 40, including embodiments that do not include any recesses or indentations along/about the stem portion 40.

In the illustrated embodiment, the distal bit 14 includes a proximal shank portion 50 and a distal engagement portion 60. In one embodiment, the proximal shank portion 50 has a non-circular shape so as to facilitate secure engagement of the distal bit 14 with the proximal handle 12 and to inhibit rotational movement of the distal bit 14 relative to the proximal handle 12. In one specific embodiment, the proximal shank portion 50 is provided with one or more flats or flattened regions 52. In another specific embodiment, the proximal shank portion 50 is hexagonally-shaped. However, other shapes and configurations of the proximal shank portion 50 are also contemplated including, for example, a star shape, a Torx shape, a square shape, a triangular shape, or other shapes suitable to inhibit rotational movement of the distal bit 14 relative to the proximal handle 12.

In the illustrated embodiment, the distal engagement portion 60 has a non-circular shape configured to facilitate rotational driving engagement of the distal bit 14 with the head of a bone screw or another type of bone anchor or orthopedic device. In one specific embodiment, the distal engagement portion 60 includes a plurality of radially-extending splines 62 extending along a length of the distal engagement portion 60. In another specific embodiment, the distal engagement portion 60 is star-shaped and is sized and shaped for receipt within a correspondingly sized/shaped driver opening in the head of the bone screw to facilitate rotational engagement of the distal bit 14 with the head of the bone screw. However, other shapes and configurations of the distal engagement portion 60 are also contemplated including, for example, a Phillips shape, a Torx shape, a hexagonal shape, a cruciform shape, a square shape, a triangular shape, a flat blade shape, or other shapes suitable to provide driving rotational engagement of the distal bit 14 with the head of the bone screw. Additionally, in some embodiments, the distal engagement portion 60 defines an inward taper in a proximal-distal direction to facilitate insertion of the distal engagement portion 60 into the driver opening in the head of the bone screw, and to provisionally and releasably engage and capture/retain the bone screw on the distal bit 14 to facilitate removal from packaging, handling between the nurse (or other medical personnel) and the surgeon, and positioning and manipulation of the bone screw to the targeted anchor location or surgical site. As should be appreciated, provisionally and releasably engaging the distal engagement portion 60 with the bone screw tends to reduce the length, complexity and overall cost of the surgical procedure.

In the illustrated embodiment, as shown in FIG. 1, the proximal handle 12 has an overall handle length lₕ and the distal bit 14 has an overall bit length l_{b} that cooperate with one another to provide the driver instrument 10 with an overall instrument length l. As should be appreciated, the overall bit length l_{b} includes a first bit length l₁ that is over molded by and encapsulated within the stem portion 40 of the proximal handle 12, and a second bit length l₂ that extends from the distal end of the stem portion 40. In one embodiment, the overall handle length lₕ is at least twice the overall bit length l_{b}. In another embodiment, the overall handle length lₕ is at least three times the overall bit length l_{b}. In a further embodiment, the overall handle length lₕ is at least four times the overall bit length l_{b}. Additionally, in some embodiments, the first bit length l₁ and the second bit length l₂ are substantially equal to one another. However, other embodiments are also contemplated where the first bit length l₁ is greater than the second bit length l₂, and still other embodiments are contemplated where the first bit length l₁ is less than the second bit length l₂.

As indicated above, in one aspect of the invention, the proximal handle 12 is made of a plastic injection molded material and is formed via a plastic injection molding process, and in another aspect of the invention, the distal bit 14 is made of a metal injection molded material and is formed via a metal injection molding process. However, it should be understood that other types of materials and formation processes or manufacturing techniques are also contemplated. In one embodiment, the distal bit 14 is initially formed via the metal injection molding process, followed by formation of the proximal handle 12 via the plastic injection molding process wherein the stem portion 40 of the proximal handle 20 is over molded about the proximal shank portion 50 of the distal bit 14. In this manner, the proximal handle 12 and the distal bit 14 are integral with one another so as to define a unitary driver instrument 10 wherein the distal bit 14 is permanently attached/engaged to the proximal handle 12. Additionally, it should be appreciated that forming the distal bit 14 via a metal injection molding process eliminates machining steps or processes commonly associated with the fabrication/manufacturing of conventional driver instruments. In some embodiments, the distal bit 14 is heat treated or hardened subsequent to the metal injection molding process to provide additional strength to the distal bit. However, the distal bit 14 does not require any significant machining steps or processes subsequent to the metal injection molding process. As should be appreciated, elimination of machining steps/processes eliminates significant manufacturing costs and provides substantial savings in the production of the distal bit 14.

In a further aspect of the invention, the orthopedic driver instrument 10 is contemplated for use in association with a single surgery or orthopedic procedure, followed by permanent disposal of the driver instrument 10. As should be appreciated, disposal of the driver instrument 10 after a single surgery or orthopedic procedure eliminates the need to clean and sterilize the driver instrument 10, which in turn reduces the overall cost associated with use of the driver instrument 10. Additionally, it should be further appreciated that the cost of producing the orthopedic driver instrument 10 is approximately one-seventh (or less than 15%) of the cost of producing a conventional/traditional orthopedic driver instrument 10. These cost reductions are the result of a significant reduction in material costs, as well as a substantial reduction in the formation/fabrication cost of producing the orthopedic driver instrument 10. Significant reductions in the cost of producing the orthopedic driver instrument 10 are realized by forming the distal bit 14 via the metal injection molding process and forming the proximal handle 12 via the plastic injection molded process, including over molding of the stem portion 40 of the proximal handle 20 about the proximal shank portion 50 of the distal bit 14.

As indicated above, the ribbed configuration of the proximal handle 12 provides the gripping portion 20 with an ergonomic non-slip configuration, and also results in a significant reduction in the amount of material required to form the proximal handle 12 without a significant reduction in the strength and structural integrity (i.e., performance) of the proximal handle 12. Additionally, the distal bit 14 satisfies high tolerance level requirements via the metal injection molding process, which further provides repeatability from part to part, thereby eliminating the manufacturing/fabrication costs normally associated with performing significant machining processes on the components of the driver instrument. Since formation of parts via a metal injection molding processes is limited to parts having a relatively short length, the overall length of the distal bit 14 is sized to be significantly less than the overall length of the proximal handle 12. As indicated above, in some embodiments, the overall handle length lₕ is at least twice the overall bit length l_{b}. In other embodiments, the overall handle length lₕ is at least three or four times the overall bit length l_{b}. The relatively shorter length of the distal bit 14 is accommodated by providing the plastic proximal handle 12 with an elongate shaft or stem portion 40 formed integral with the main body 20 of the proximal handle 12 via the plastic injection molding process, and with the elongate stem portion 40 over molded about the proximal end portion of the distal bit 14 to form an integral driver instrument. As should be appreciated, conventional/traditional driver instruments typically include metallic drive shafts that have a significantly greater length compared to the much shorter length of the driver bit 14, thereby precluding formation of the metallic drive shaft of conventional/traditional driver instruments by way of a metal injection molding process.

In some embodiments, the orthopedic driver instrument 10 may be provided as a stand-alone instrument. However, in other embodiments, the orthopedic driver instrument 10 may be provided in a kit including an orthopedic support element such as, for example, a bone plate, along with a plurality of bone anchors such as, for example, bone screws.

Referring once again to FIG. 7, in some embodiments, the orthopedic driver instrument 10 may include indexing markings or indicia 70 positioned along one or more regions of the proximal handle 12 and/or the distal driver bit 14. As will be discussed in detail below, the indexing markings or indicia 70 provide the surgeon or other medical personnel with a visual or tactile indication as to rotational displacement of the driver instrument 10 to manually control or limit the driving torque applied to the bone screw or fastener being driven into bone tissue by the driver instrument 10. As should be appreciated, controlling or limiting the amount of torque applied to the bone screw or fastener minimizes risks associated with overtightening and/or undertightening of the bone screw including, for example, stripping of the internal threads in the bone tissue, damage to associated implants or other structures to which the bone screw is attached, and/or loosening or back out of the bone screw from the bone tissue. Although the indexing markings 70 have been illustrated in association with the embodiment of the driver instrument 10 shown in FIG. 7, it should be understood that the indexing markings 70 may likewise be used in association with the embodiment of the driver instrument 10 shown in FIGS. 1-6, or with other non-illustrated embodiments of the driver instrument 10.

In the illustrated embodiment of the driver instrument 10, the indexing markings 70 are positioned along the central and distal regions 40b, 40c of the stem portion 40. However, it should be appreciated that the indexing markings 70 may be positioned along other regions/portions and at other locations of the proximal handle 12 and/or the distal driver bit 14. For example, indexing markings 70 may be positioned along the proximal transition region 40a of the stem portion 40, and/or along any region of the gripping portion 20 of the proximal handle 12 including, for example, along the ribs 30, 32 and/or the non-ribbed region 36 of the gripping portion 20. The driver instrument embodiment illustrated in FIG. 7 includes indexing markings or indicia 70 positioned along the central region 20c of the gripping portion 20, although providing indexing markings 70 along the proximal region 20a and/or the distal region 20c of the gripping portion 20 is also contemplated. Additionally, in still other embodiments, indexing markings 70 may be positioned along the proximal shank portion 50 and/or the distal engagement portion 60 of the distal driver bit 14.

In the illustrated embodiment of the driver instrument 10 in FIG. 7, the indexing markings 70 are provided as lines or stripes 70a and raised bumps or protrusions 70b. In the illustrated embodiment, the lines/stripes 70a are provided as thick, black, solid and continuous linear markings. However, in other embodiments, the lines/stripes 70a may have other thickness (i.e., thin/narrow lines), may be provided in other colors (i.e., red, white, etc.), may be provided with a divided configuration (i.e., a double line), may be provided with a discontinuous configuration (i.e., a dashed or broken line), may be provided with a non-linear configuration (i.e., a curved or curvilinear line, circular shapes or dots), or may have any other suitable configuration to provide a visually perceptible indication as to the rotational position and/or rotational displacement of the driver instrument 10. Additionally, in the illustrated embodiment, the raised bumps or protrusions 70b are provided as hemispherical-shaped circular protrusions. However, in other embodiments, the raised bumps or protrusions 70b may have other shapes and configurations (i.e., star, square, rectangular, triangular, polygonal, elliptical, ovular, etc.), may be colorless or may be provided in a variety of different colors, or may have any other suitable configuration to provide a tactilely (and possibly visually) perceptible indication as to the rotational position and/or rotational displacement of the driver instrument 10.

It should be appreciated that other types of indexing markings or indicia are also contemplated for use in association with the driver instrument 10 including, for example, dots or circular shapes, arrows, non-linear shapes, symbols, letters, numbers, colors, or any other visually or tactilely perceptible marking or indicia. Additionally, it should be appreciated that the indexing marking or indicia 70 may be provided as laser markings or etchings, printed markings, painted markings, silk screened markings, inscriptions, engravings, grooves, recesses, depressions, impressions, raised features, colorations, discolorations, or any other suitable marking or indicia to provide a visually or tactilely perceptible indication as to the rotational position and/or rotational displacement of the driver instrument 10.

In the illustrated embodiment, each set, pair or group 70a, 70b of the indexing markings or indicia 70 includes at least two markings/indicia that are angularly offset from one another relative to the longitudinal axis L about a circumference of the driver instrument 10. In one embodiment, the indexing markings 70a, 70b are each provided in pairs of indexing markings positioned on opposite sides of the driver instrument 10. It is noted that only one of the indexing markings 70a, 70b of each pair is illustrated in FIG. 7, it being understood that another marking/indicia is positioned on the opposite side of the driver instrument 10. In other words, the pairs of indexing markings 70a, 70b are offset from one another by about 180 degrees and are positioned on opposite sides of the driver instrument 10. However, it should be understood that in other embodiments, each set/pair/group of the indexing markings 70a, 70b may include any number of markings/indicia including a single marking, three markings, or four or more markings positioned about a circumference of the driver instrument 10, and preferably angularly offset from one another in a substantially uniform manner (i.e., three indexing markings angularly offset by 120°, four indexing markings angularly offset by 90°, etc.). It should also be understood that the indexing markings or indicia 70 of a set/pair/group need not necessarily be positioned at the same axial location along the longitudinal axis L, but may instead by axially offset from one another along the longitudinal axis L.

In some embodiments, each of the indexing markings 70 of a set/pair/group may be of the same type/configuration (i.e., the indexing markings of a set/pair/group are configured identical to one another). However, in other embodiments, the indexing markings 70 of a set/pair/group may have different types/configurations or may be provided with different distinguishing features or characteristics to facilitate visual or tactile recognition of the particular rotational position or rotational displacement of the driver instrument 10 during driving of a screw or fastener into bone tissue. For example, in one embodiment, the indexing markings 70a may include a solid line on one side of the driver instrument (as shown in FIG. 7) and a divided/double line on the opposite side of the driver instrument to provide a degree of differentiation if desired to indicate different rotational positions or displacement of the driver instrument 10. In other embodiments, the indexing markings 70a may include a thick line on one side of the driver instrument (as shown in FIG. 7) and a thinner line on the opposite side of the driver instrument, a continuous line on one side of the driver instrument (as shown in FIG. 7) and a dashed or broken line on the opposite side of the driver instrument, a black line on one side of the driver instrument (as shown in FIG. 7) and a line of a different color on the opposite side of the driver instrument. Similarly, the indexing markings/indicia 70b may include a circular bump/protrusion on one side of the driver instrument (as shown in FIG. 7) and a bump/protrusion having a different shape/configuration on the opposite side of the driver instrument to provide a degree of differentiation if desired to indicate different rotational positions or displacement of the driver instrument 10. In other embodiments, the indexing markings 70b may include a bump/protrusion of a first color on one side of the driver instrument and a bump/protrusion of a different color on the opposite side of the driver instrument.

In still other embodiment, one of the indexing markings/indicia 70 of a set/pair/group may be of a first type or have a first feature/characteristic, and at least one other of the indexing markings/indicia 70 may be of a second type or have a second feature/characteristic that is visually or tactilely distinguishable from the first type. The distinguishing type/feature/characteristic may be different colors, shapes, symbols, letters, numbers, or any other visually distinguishable type, feature or characteristic. In one embodiment, at least one of the indexing markings/indicia 70 may be provided with a first color (i.e., red), and at least one of the indexing markings/indicia 70 may be provided with a different second color (i.e., black or blue). In one exemplary embodiment, two of the indexing markings/indicia positioned generally diametrically opposite one another may be provided with a first color (i.e., red), and two of the indexing markings/indicia positioned generally diametrically opposite one another may be provided with a different second color (i.e., black or blue). In another embodiment, at least one of the indexing markings/indicia may have a first shape (i.e., a dot) and at least one other of the indexing markings/indicia 70 may have a different second shape (i.e., a dash/line). Additionally, in another exemplary embodiment, the indexing markings/indicia 70 of a set/pair/group may have alternating or staggered types/features/characteristics such that every other indexing marking/indicia 70 has an alternating type/feature/characteristic (i.e., red-blue-red-blue or dot-dash-dot-dash, etc.). In still another exemplary embodiment, the indexing markings/indicia 70 of a set/pair/group may have sequential features/characteristics to indicate sequential rotational positions or displacement of the driver instrument 10 (i.e., 1-2-3-4 or A-B-C-D, etc.). As indicated above, providing the indexing markings or indicia 70 with distinguishing features or characteristics may promote visual or tactile recognition of the degree of angular displacement of the driver instrument 10 during driving of a screw or fastener into bone tissue.

As indicated above, the indexing markings or indicia 70 provide the surgeon or other medical personnel with a visual and/or tactile indication as to rotational position or rotational displacement of the driver instrument 10 to manually control or limit the driving torque applied to the bone screw or fastener being driven into bone tissue by the driver instrument 10. In one embodiment, as the bone screw or fastener is being driven into bone tissue by the driver instrument 10, at a point prior to the bone screw being fully driven or engaged in the bone tissue, the surgeon is provided with a provisional indication or feedback that the bone screw is approaching or near its fully engaged or locked state. In one embodiment, the provisional indication or feedback may be provided when a lower surface of the screw head (or another portion of the screw) engages or abuts a corresponding surface on a bone plate or another type of orthopedic implant. In one exemplary embodiment, the surgeon may be provided with a “tactile feel” associated with engagement of the screw head (or another portion of the screw) with another feature associated with an implant or device. However, in other embodiments, the provisional indication or feedback may be provided via a visual or audible indication (i.e., via a visual alignment of one structural feature relative to another structural feature, or via a sound generated by engagement of one structural feature with another structural feature).

Once the provisional indication/feedback is received or perceived by the surgeon, the driver instrument 10 (and the bone screw) is rotated or indexed an additional predetermined amount/degree to the fully engaged or locked state of the bone screw or fastener. As should be appreciated, the additional predetermined amount/degree of rotational or angular displacement may be measured by the indexing markings or indicia 70, 70a, 70b. For example, in one embodiment, the additional predetermined amount/degree of rotational or angular displacement may be one-half turn or 180° of additional rotational displacement, as measured by the angular passage of a certain number of the indexing markings or indicia 70, 70a, 70b from a selected reference position or location. In other embodiments, the additional predetermined amount/degree of rotational or angular displacement may be one-quarter turn or 90° of additional rotational displacement, three-quarter turn or 270° of additional rotational displacement, or full turn or 360° of additional rotational displacement, as measured by the angular passage of a certain number of the indexing markings or indicia 70, 70a, 70b from a selected reference position or location. However, it should be understood that the additional predetermined amount/degree of rotational or angular displacement may vary and is not limited to the exemplary embodiments set forth above.

In the embodiment of the driver instrument 10 illustrated in FIG. 7, the visually-perceptible indexing markings or indicia 70a include two indexing markings/indicia positioned on opposite sides of the driver instrument 10. Once the provisional indication/feedback is received or perceived by the surgeon, the driver instrument 10 (and the bone screw) may be rotated or indexed an additional one-half turn or 180° to the fully engaged or locked state of the bone screw or fastener, as measured by rotation of the driver instrument 10 until the marking/indicia 70a on the opposite side of the driver instrument is positioned at or near the original angular position of the other marking/indicia 70a. However, as indicated above, the driver instrument 10 may be provided with three or more of the indexing markings/indicia 70a to provide additional resolution or gradations to accommodate other degrees of rotational displacement or indexing from the initial rotational position to the fully engaged or locked rotational position of the bone screw or fastener.

Additionally, in the embodiment of the driver instrument 10 illustrated in FIG. 7, the tactilely-perceptible indexing markings or indicia 70b include two indexing markings/indicia positioned on opposite sides of the driver instrument 10. Once the provisional indication/feedback is received or perceived by the surgeon, the driver instrument 10 (and the bone screw) may be rotated or indexed an additional one-half turn or 180° to the fully engaged or locked state of the bone screw or fastener, as measured by rotation of the driver instrument 10 until the marking/indicia 70b on the opposite side of the driver instrument is positioned at or near the original angular position of the other marking/indicia 70b. However, as indicated above, the driver instrument 10 may be provided with three or more of the indexing markings/indicia 70b to provide additional resolution or gradations to accommodate other degrees of rotational displacement or indexing from the initial rotational position to the fully engaged or locked rotational position of the bone screw or fastener. With regard to the tactilely-perceptible indexing markings or indicia 70b, the surgeon may use one hand (i.e., the right hand) to grasp the gripping portion 20 of the proximal handle 14 to rotate the driver instrument 10 and drive the screw/fastener, and may use one or more fingers of the other hand (i.e., the left hand) to provide a tactile feel of the indexing markings/indicia 70b to determine the rotational position or displacement of the driver instrument 10 between the initial rotational position to the fully engaged or locked rotational position of the bone screw or fastener.

As should be appreciated, the indexing markings/indicia 70, 70a, 70b associated with the orthopedic driver instrument 10 provide the surgeon with a visual or tactile indication as to the appropriate amount of additional rotational or angular displacement of the driver instrument 10 from an initial rotational position (i.e., the rotational position at which a provisional indication/feedback is received or perceived by the surgeon) to a final rotational position corresponding to the fully engaged or locked state of the bone screw or fastener, thereby minimizing the negative effects and potential risks associated with overtorquing, overtightening and/or undertightening the bone screw or fastener.

While the instruments and methods set forth above have been described in association with an orthopedic driver instrument for use in surgeries or other orthopedic procedures, it should be understood that the instruments and methods may also be used in other technological areas and/or in association with other types of instruments. In reading the claims, words such as “a”, “an”, “at least one”, and “at least a portion” are not intended to limit the claims to only one item unless specifically stated to the contrary. Additionally, when the language “at least a portion” and/or “a portion” is used, the claims may include a portion and/or the entire item unless specifically stated to the contrary. Furthermore, when the term “distal” is used with respect to a structure, the term refers to the far end of the structure, and when the term “proximal” is used with respect to a structure, the term refers to the near end of the structure.

Various changes and modifications to the described embodiments described herein will be apparent to those skilled in the art, and such changes and modifications can be made without departing from the spirit and scope of the invention and without diminishing its intended advantages. Additionally, while the invention has been illustrated and described in detail in the drawings and foregoing description, the same is to be considered illustrative and not restrictive in character, it being understood that only selected embodiments have been shown and described and that all changes, equivalents, and modifications that come within the scope of the inventions described herein or defined by the following claims are desired to be protected.

What is claimed is:

1. A method of producing an orthopedic driver instrument, comprising:
forming a metallic driver bit using a metal injection molding process; and
forming a plastic handle using a plastic injection molding process, the plastic handle having a gripping portion and an elongate shaft portion extending axially from the gripping portion, wherein the plastic injection molding process comprises over molding the elongate shaft portion of the plastic handle about a shank portion of the metallic driver bit.

2. The method of claim 1, wherein the forming of the plastic handle comprises providing the gripping portion with a longitudinally extending concave surface contour.

3. The method of claim 2, wherein the forming of the plastic handle comprises providing the gripping portion with a longitudinally extending convex surface contour on each side of the longitudinally extending concave surface contour.

4. The method of claim 1, wherein the forming of the plastic handle comprises providing the gripping portion with a plurality of longitudinally extending ribs and a plurality of transversely extending ribs extending between adjacent pairs of the longitudinally extending ribs.

5. The method of claim 1, wherein the metallic driver bit has an overall bit length; and
wherein the plastic handle has an overall handle length that is at least twice the overall bit length.

6. The method of claim 5, wherein the overall handle length is at least three times the overall bit length.

7. The method of claim 6, wherein the overall handle length is at least four times the overall bit length.

8. The method of claim 1, wherein the gripping portion of the plastic handle defines a first outer cross section and the elongate shaft portion of the plastic handle defines a second outer cross section smaller than the first outer cross section.

9. The method of claim 1, further comprising using the orthopedic driver instrument in a single surgery; and
permanently disposing of the orthopedic driver instrument after the single surgery.

10. The method of claim 1, further comprising providing a plurality of indexing markings or indicia on an outer surface of the plastic handle and/or the metallic driver bit to provide a visual or tactile indication as to a rotational position or rotational displacement of the orthopedic driver instrument to manually control or limit driving torque applied to a bone screw or fastener driven by the orthopedic driver instrument.

11. The method of claim 10, wherein the plurality of indexing markings or indicia comprise visual markings positioned at different angular positions about the outer surface to provide a visual indication as to the rotational position or rotational displacement of the orthopedic driver instrument.

12. The method of claim 11, wherein the visual markings comprise at least one of a line, dash, dot, shape, color, number, letter or symbol.

13. The method of claim 10, wherein the plurality of indexing markings or indicia comprise tactile indicia positioned at different angular positions about the outer surface to provide a tactile indication as to the rotational position or rotational displacement of the orthopedic driver instrument.

14. The method of claim 13, wherein the tactile indicia comprise at least one of a raised bump, projection, protrusion, recess, depression, impression, indentation or groove.

15. A method of producing an orthopedic driver instrument, comprising:
forming a metallic driver bit using a metal injection molding process, the metallic driver bit having an overall bit length; and
forming a plastic handle over a portion of the metallic driver bit using a plastic injection molding process, the plastic handle having an overall handle length that is at least twice the overall bit length.

16. The method of claim 15, wherein the overall handle length is at least three times the overall bit length.

17. The method of claim 16, wherein the overall handle length is at least four times the overall bit length.

18. The method of claim 17, wherein the forming of the plastic handle comprises:
providing the gripping portion with a longitudinally extending concave surface contour; and
providing the gripping portion with a longitudinally extending convex surface contour on each side of the longitudinally extending concave surface contour.

19. The method of claim 15, further comprising using the orthopedic driver instrument in a single surgery; and
permanently disposing of the orthopedic driver instrument after the single surgery.

20. An orthopedic driver instrument, comprising:
a metal injection molded driver bit having an overall bit length; and
a plastic injection molded handle having an overall handle length that is at least twice the overall bit length, the handle including a gripping portion and an elongate shaft portion extending axially from the gripping portion, the elongate shaft portion over molded about a shank portion of the metallic driver bit.

21. The orthopedic driver instrument of claim 20, wherein the overall handle length is at least three times the overall bit length.

22. The orthopedic driver instrument of claim 21, wherein the overall handle length is at least four times the overall bit length.

23. The orthopedic driver instrument of claim 20, wherein the gripping portion of the handle defines a first outer cross section and the elongate shaft portion of the handle defines a second outer cross section smaller than the first outer cross section.

24. The orthopedic driver instrument of claim 20, wherein the gripping portion of the handle defines a longitudinally extending concave surface contour; and
wherein the gripping portion of the handle defines a longitudinally extending convex surface contour on each side of the longitudinally extending concave surface contour.

25. The orthopedic driver instrument of claim 20, wherein the gripping portion of the handle comprises a plurality of longitudinally extending ribs and a plurality of transversely extending ribs extending between adjacent pairs of the longitudinally extending ribs.

26. The orthopedic driver instrument of claim 20, further comprising a plurality of indexing markings or indicia positioned on an outer surface of the handle and/or the driver bit to provide a visual or tactile indication as to a rotational position or rotational displacement of the orthopedic driver instrument to manually control or limit driving torque applied to a bone screw or fastener driven by the orthopedic driver instrument.

27. The orthopedic driver instrument of claim 26, wherein the plurality of indexing markings or indicia comprise visual markings positioned at different angular positions about the outer surface to provide a visual indication as to the rotational position or rotational displacement of the orthopedic driver instrument.

28. The orthopedic driver instrument of claim 27, wherein the visual markings comprise at least one of a line, dash, dot, shape, color, number, letter or symbol.

29. The orthopedic driver instrument of claim 26, wherein the plurality of indexing markings or indicia comprise tactile indicia positioned at different angular positions about the outer surface to provide a tactile indication as to the rotational position or rotational displacement of the orthopedic driver instrument.

30. The orthopedic driver instrument of claim 29, wherein the tactile indicia comprise at least one of a raised bump, projection, protrusion, recess, depression, impression, indentation or groove.
